(19) United States
(12) Patent Application Publication
(10) Pub. No.: US 2007/0117996 A1
(43) Pub. Date: May 24, 2007

(54) PROCESS FOR PREPARING SIMVASTATIN

(76) Inventors: Srinivasulu Gudipati, Hyderabad 500 072, A.P. (IN); Venkata Naga Brahmeswara Rao Mandava, Hyderabad 500 072, A.P. (IN); Radha Krishna Singam Setty, Hyderabad 500 072, A.P. (IN); Nagaraju Manne, Nalgonda 508 277, A.P. (IN); Rajeshwar Reddy Sagyam, Hyderabad 500 072, A.P. (IN); Satish Kumar Vujjini, Hyderabad 500 070, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/561,599

(22) Filed: Nov. 20, 2006

Related U.S. Application Data
(60) Provisional application No. 60/746,212, filed on May 2, 2006.

(30) Foreign Application Priority Data
Nov. 21, 2005 (IN) 1691/CHE/2005

(51) Int. Cl. C07C 67/02

(52) U.S. Cl. 560/256

(57) ABSTRACT

A process for preparing simvastatin, intermediate compounds, and a solid premix comprising simvastatin and butylated hydroxyanisole.

The present invention relates to a process for the preparation of simvastatin and intermediates thereof.

Simvastatin has the chemical name butanoic acid, 2,2-dimethyl-, 1,2,3,7,8,8a-hexahydro-3,7-dimethyl-8-[2-(tetrahydro-4-hydroxy-6-oxo-2H-pyran-2-yl)-ethyl]-naphthalenyl ester, [1S-[1α,3α,7β,8β(2S*,4S*),-8aβ]], hereinafter referred to by the adopted name “simvastatin,” and is structurally represented in Formula I.

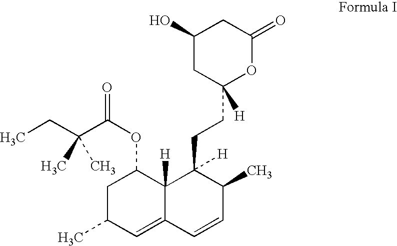

Simvastatin is a potent anti-hypercholesterolemic agent. It inhibits the enzyme 3-hydroxy-3-methyl-glutaryl coenzyme A reductase (“HMG-CoA reductase”), which catalyzes the formation of mevalonic acid, and thus inhibits cholesterol biosynthesis. It also increases the number of cellular LDL-receptors that remove the LDL cholesterol circulating in the blood, and thereby lower blood cholesterol levels. Pharmaceutical products containing the compound are commercially available under the trade name ZOCOR™.

U.S. Pat. No. 4,444,784 discloses simvastatin, its pharmaceutically acceptable salts, pharmaceutical compositions and their use in the treatment of hypercholesterolemia. It also discloses a process for the synthesis of simvastatin, which involves deacylation of lovastatin followed by a subsequent acylation with the 2,2-dimethylbutyryl moiety.

Subsequently several processes have been described for preparation of simvastatin.

U.S. Pat. No. 6,472,542 describes the process for the preparation of simvastatin, which comprises protecting the free hydroxyl groups of the lovastatin amide with hexamethyldisilazane (HMDS).

U.S. Pat. No. 6,271,398 describes the process for the preparation of simvastatin by protecting the two free hydroxyl groups of the lovastatin amide with an ether group.

It remains desirable to provide a simple, industrially feasible, inexpensive, and scaleable process for the synthesis of simvastatin of Formula I.

### SUMMARY OF THE INVENTION

The present invention relates to a process for the preparation of simvastatin and intermediates thereof.

In one aspect the present invention provides a process for the preparation of simvastatin comprising:

i) protecting the free hydroxyl group of the compound lovastatin of Formula II 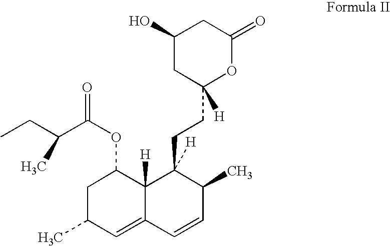
with 3,4-dihydro-2H-pyran to afford the hydroxy-protected lovastatin compound of Formula III 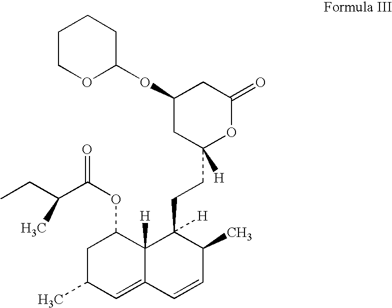
and, without isolating the compound of Formula III, forming an amide of lovastatin, thereby opening the lactone ring of the lovastatin to form a protected lovastatin amide of Formula IV;

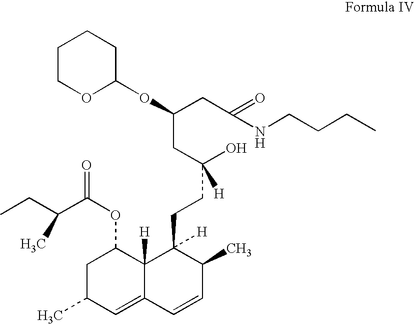

ii) methylating the α-carbon of the 2-methylbutyrate side chain of the protected lovastatin amide to form protected simvastatin amide of Formula V;

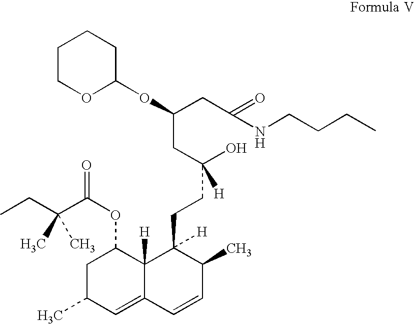

iii) removing the tetrahydropyran protecting group from compound of Formula V by reacting with an acid to afford simvastatin amide of Formula VI;

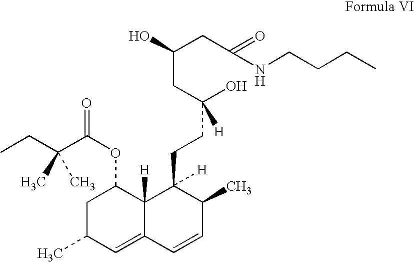

iv) hydrolyzing the simvastatin amide of Formula VI to form simvastatin acid of Formula VII 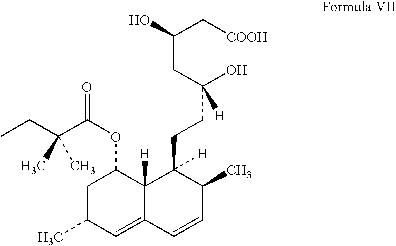
and, without isolating the compound of Formula VII, forming a simvastatin ammonium salt of Formula VIII in an intermediate step; and

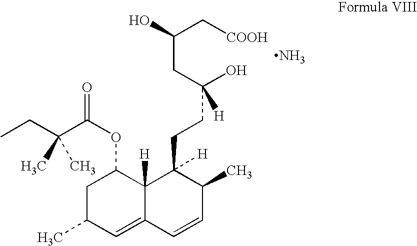

v) forming a lactone ring in the simvastatin ammonium salt of Formula VIII to form simvastatin of Formula I.

In other aspects, the present invention provides a hydroxy-protected lovastatin compound of Formula III, lovastatin amide of Formula IV and protected simvastatin amide of Formula V, and processes for their preparation.

An embodiment of the invention includes a process for preparing simvastatin, comprising reacting a compound having a formula: 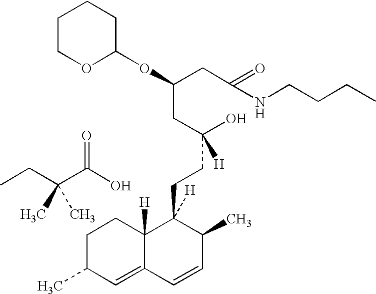
with an acid to form a compound having a formula:

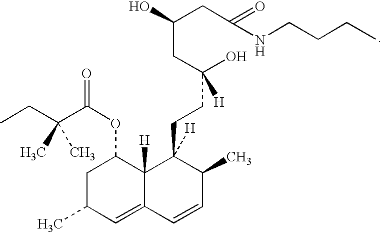

Another embodiment of the invention includes a process for preparing simvastatin, comprising methylating a compound having a formula: 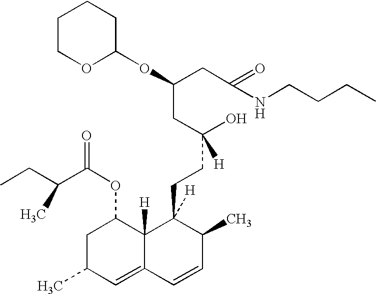
to form a compound having a formula:

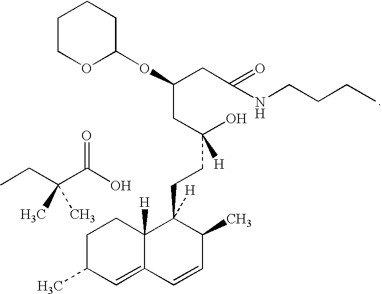

A further embodiment of the invention includes a process for preparing simvastatin, comprising reacting lovastatin with 3,4-dihydro-2H-pyran to form an intermediate having a formula: 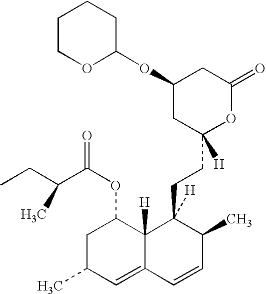
and, without isolating an intermediate, reacting with an amine to form a compound having a formula:

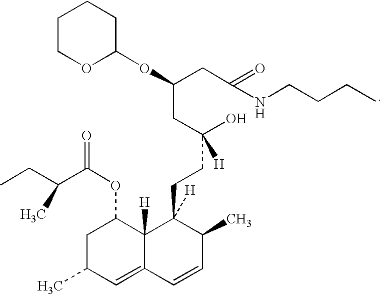

In an additional embodiment, the invention includes a process for preparing simvastatin, comprising reacting lovastatin with 3,4-dihydro-2H-pyran to form an intermediate having a formula: 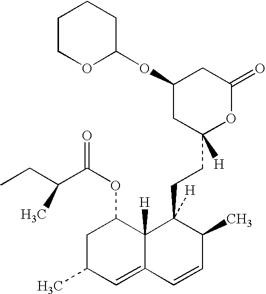
and, without isolating an intermediate, reacting with an amine to form a compound having a formula: 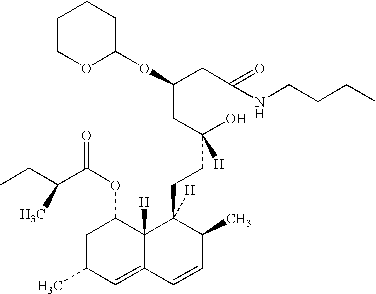
then methylating to form a compound having a formula: 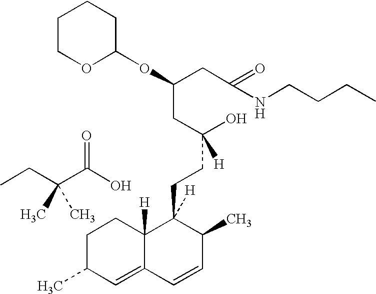
and reacting with an acid to form a compound having a formula:

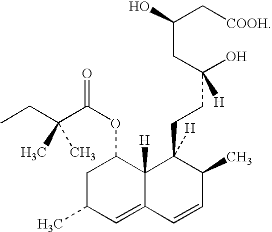

In a further embodiment, the invention includes a solid premix comprising simvastatin and butylated hydroxyanisole.

In a still further embodiment, the invention includes a process for preparing a solid premix, comprising combining a solution of simvastatin with butylated hydroxyanisole and water.

### DETAILED DESCRIPTION OF THE INVENTION

The present invention relates to a process for the preparation of simvastatin and intermediates thereof.

In one aspect the present invention provides a process for the preparation of simvastatin comprising:

i) protecting the free hydroxyl group of the lovastatin of Formula II 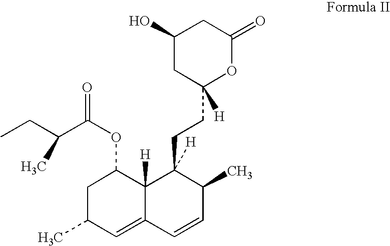
with 3,4-dihydro-2H-pyran to afford 2-methyl-butyric acid 3,7-dimethyl-8-{2-[6-oxo-4-(tetrahydro-pyran-2-yloxy)-tetrahydro-pyran-2-yl]-ethyl}-1,2,3,7,8,8a-hexahydro-naphthalen-1-yl ester (“hydroxy-protected lovastatin”) of Formula III 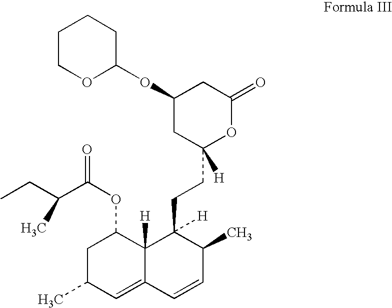
and, without isolating the compound of Formula III, forming an amide of lovastatin, thereby opening the lactone ring of the lovastatin to form 2-methyl-butyric acid 8-[6-butyl carbamoyl-3-hydroxy-5-(tetrahydro-pyran-2-yloxy)-hexyl]-3,7-dimethyl-1,2,3,7,8,8a-hexahydro-naphthalen-1-yl ester (“protected lovastatin amide”) of Formula IV;

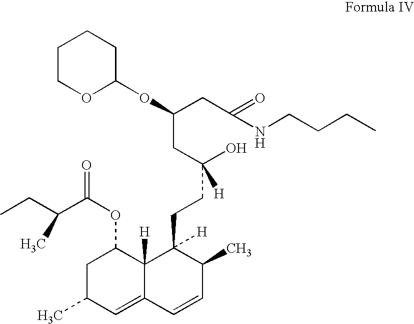

ii) methylating the α-carbon of the 2-methylbutyrate side chain of the protected lovastatin amide to form 2,2-dimethyl-butyric acid 8-[6-butylcarbamoyl-3-hydroxy-5-(tetrahydro-pyran-2-yloxy)-hexyl]-3,7-dimethyl-1,2,3,7,8,8a-hexahydro-naphthalen-1-yl ester (“protected simvastatin amide”) of Formula V;

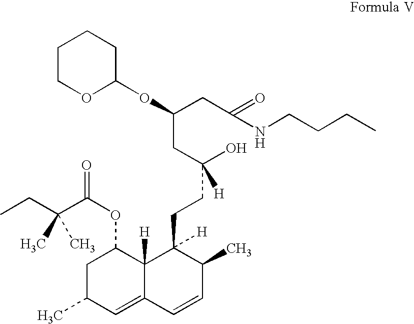

iii) removing the tetrahydropyran-protecting group from the compound of Formula V by reacting with an acid to afford 2,2-dimethyl-butyric acid 8-(6-butylcarbamoyl-3,5-dihydroxy-hexyl)-3,7-dimethyl-1,2,3,7,8,8a-hexahydro-naphthalen-1-yl ester (“simvastatin amide”) of Formula VI;

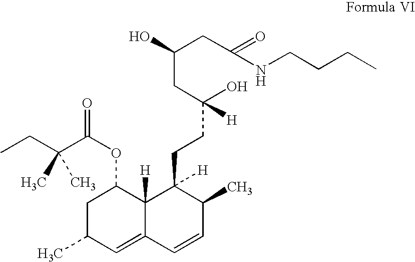

iv) hydrolyzing the simvastatin amide of Formula VI to form simvastatin acid of Formula VII 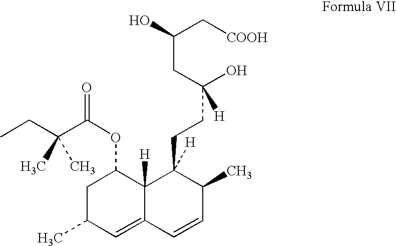
and, without isolating the compound of Formula VII, forming a simvastatin ammonium salt of Formula VIII in an intermediate step; and

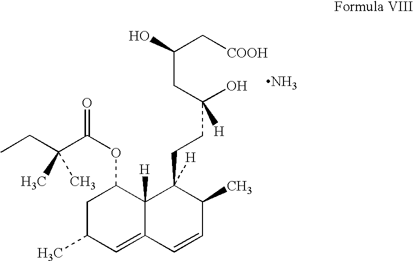

v) forming a lactone ring in the simvastatin ammonium salt of Formula VIII to form simvastatin of Formula I.

Step i) involves protection of the free hydroxyl group of the lovastatin of Formula II with 3,4-dihydro-2H-pyran in the presence of a suitable solvent and an acid to give the protected lovastatin compound of Formula III, then without isolating the compound of Formula III forming an amide of lovastatin, thereby opening the lactone ring of the lovastatin to form a lovastatin amide of Formula IV;

Suitable amounts of 2,3-dihydropyran that can be used in the present invention ranges from about 1 to about 5 molar equivalents per molar equivalent of lovastatin of Formula II

Suitable acids that can be used include but are not limited to organic and inorganic acids such as: organic acids including formic acid, acetic acid, p-toluene sulfonic acid and the like; inorganic acids including sulphuric acid, hydrochloric acid and the like; and/or mixtures thereof or their combinations in various proportions with water.

Suitable amounts of acid that can be used in the present invention ranges from about 1 to about 5 molar equivalent per molar equivalent of lovastatin of Formula II. Suitably the acid that is used in the present invention can be used in its natural form, or be adsorbed on silica gel prior to use.

Suitable solvents that can be used for the protection of the hydroxy group include but are not limited to: ketones such as acetone, ethyl methyl ketone, methyl isobutyl ketone, and the like; esters such as ethyl acetate, n-propyl acetate, n-butyl acetate, t-butyl acetate, and the like; nitriles such as acetonitrile, priopionitrile, and the like; ethers such as diethyl ether, dimethyl ether, tetra hydrofuran and the like; halogenated hydrocarbons such as dichloromethane, ethylene dichloride, chloroform, and the like; aprotic solvents such as N,N-dimethyl formamide (DMF), dimethylsulfoxide (DMSO), N,N-dimethyl acetamide (DMA), and the like; and mixtures thereof or their combinations in various proportions.

Suitable temperatures for conducting the reaction can range from −5 to 45° C., or from about 0 to 35° C., or from about 2 to 35° C., or the reflux temperature of the solvent used, either at atmospheric pressure or with a different pressure. The duration of maintenance of the reaction mixture at the reaction temperatures for reaction completion vary considerably depending on the reactants and conditions chosen, for example about 1 to 48 hours, or about 1 to 36 hours, or about 5 to 24 hours, or longer.

The protected lovastatin of Formula III obtained by the process of the present invention can be isolated or it can be used in the next stage without isolation. Isolation can be accomplished by removing the solvent, adding an antisolvent to precipitate the compound, solvent extraction, or other techniques.

The protected lovastatin of Formula III is then converted to lovastatin amide by opening the lactone ring to afford the compound of protected lovastatin amide of Formula IV by reacting with an amine in the presence of a suitable solvent.

Suitable amines that can be used for the formation of the lovastatin amide of Formula IV include, without limitation, primary amines having formula RNH₂ wherein R represents a straight or branched alkyl group of 1 to 8 carbon atoms, a cycloalkyl group of 3 to 7 carbon atoms, or an aralkyl group having 1 to 6 carbon atoms in the alkyl chain. Typically, the aralkyl group contains 1 to 4 carbon atoms in the alkyl moiety, although such is not required, and the aromatic moiety is phenyl or naphthyl. Examples of suitable R groups without limitation include methyl, ethyl, propyl (iso- and n-forms), butyl (tert-, iso- and n-forms), cyclohexyl, cyclopentyl, benzyl, phenethyl, and 3-phenylpropyl.

In one embodiment of the present invention, n-butyl amine is used for the formation of lovastatin amide of Formula IV.

Suitable solvents that can be used for the formation of lovastatin amide of Formula IV include but are not limited to inert solvents such as: ethers such as diethyl ether, disiopropyl ether, tetrahydrofuran, 1,4-dioxane, and the like; aromatic hydrocarbons such as toluene, xylene and the like; nitriles such as acetonitrile, propionitrile, and the like; protic solvents such as dimethyl formamide, dimethyl acetamide, and the like; and mixtures thereof.

Suitable temperatures for conducting the reaction range from 0 to 70° C., or from about 5° C. to 65° C., or the reflux temperature of the solvent used either at atmospheric pressure or with a different pressure.

Suitably lovastatin amide of Formula IV is isolated by removing the solvent from the reaction mixture.

Solvent can be removed using conventional techniques like distillation, evaporation and the like.

In one embodiment of the present invention lovastatin amide for Formula IV is isolated by subjecting the reaction mass to distillation to remove the solvent.

Suitable techniques which can be used for the distillation include, heating the reaction mixture to reflux or by distillation using a rotational evaporator device such as a Buchi Rotavapor, spray drying, agitated thin film drying (“ATFD”), and the like.

Distillation can be conducted under vacuum, at elevated temperatures such as about 20° C. to about 70° C. Any temperature and vacuum conditions can be used as long as there is no increase in the impurity levels of the product.

Step ii) involves methylating the α-carbon of the 2-methylbutyrate side chain of the protected lovastatin amide to form protected simvastatin amide of Formula V.

Methylation of the protected lovastatin amide can be carried out by first forming an anion according to known methods, e.g., reacting an alkali metal amide in inert organic solvent with a protected lovastatin amide of Formula IV which is itself dissolved in an organic solvent medium, followed by adding an alkylating agent, e.g., a methyl halide. Methylation can be carried out at atmospheric pressure.

The alkali metal amide used in methylation can be prepared by reacting a secondary amine, e.g., pyrrolidine, piperidine or a dialkylamine, with an n-butyl- or an n-hexyl-alkali metal compound, such as n-hexyllithium or n-butyllithium, in an anhydrous ether solvent medium, e.g. tetrahydrofuran, diethyl ether or 1,2-dimethoxyethane, at a temperature of about −10° C. to −60° C., or from about −25° C. to about −30° C. In an embodiment, the alkali metal amide is prepared by a combination of n-butyllithium with a lower secondary amine, e.g., pyrrolidine or piperidine, in an ether solvent, e.g. tetrahydrofuran, at a low temperature (−10° C. to −40° C.). When the alkali metal amide, e.g., lithium pyrrolidide, is formed it is added to an organic solvent solution of a protected lovastatin amide to form an anion by enolizing the protected lovastatin amide anion. Regarding the proportion of anion forming reactants, the amount of alkali metal amide added to the protected lovastatin amide can be about 5 moles to about 15 moles of alkali metal amide per mole of protected lovastatin amide.

The methylating agent can be a methyl halide, such as methyl iodide, methyl chloride, methyl bromide, dimethyl sulphide and the like. Regarding proportions, the amount of methyl iodide (>about 99% pure), for example, added to the protected lovastatin amide anion is about 3 to about 10 molar equivalents per mole of protected lovastatin amide. In an embodiment, the methylation reaction is performed in an inert atmosphere, e.g., under nitrogen, in an anhydrous ether solvent medium, e.g. tetrahydrofuran, diethyl ether or 1,2-dimethoxyethane.

Suitable inert solvents that can be used for methylation includes but are not limited to any solvent or mixture of solvents in which the required compounds are soluble. Examples include: ethers such as dimethyl ether, diethyl ether, di-isopropyl ether, methyl tertiary-butyl ether, tetrahydrofuran, 1,4-dioxane, and the like; aliphatic hydrocarbons such as C₁-C₁₀ straight chain or branched hydrocarbons, and the like; aromatic hydrocarbons such as toluene, xylene, and the like; and mixtures thereof.

Suitable temperatures for methylation can range from −10 to −70° C., or from about −20 to −60° C., or from about −30° C. to −50° C., either at atmospheric pressure or at a different pressure.

After the completion of the methylation, the reaction mixture is quenched by adding water.

Water can be added to the reaction mass at a temperature from about −10° C. to 10° C.

Water quantities that can be used in the reaction can range from about 5 to about 15 times the weight of protected lovastatin amide used in the reaction.

Suitably, quenching can be done by adding water to the reaction mass or by adding the reaction mass to the water.

Quenching is carried out to in order to control levels of the base and the solvent, which makes the reaction highly exothermic.

The protected simvastatin amide of Formula V of the present invention is isolated by removing the solvent from the organic phase.

The solvent that is used in the present in can be removed by using conventional techniques like distillation, evaporation and the like.

In particular in the present invention protected simvastatin amide of Formula V is isolated by subjecting the reaction mixture to distillation and removing the solvent.

Suitable techniques which can be used for the distillation include, heating the reaction mixture to reflux or by distillation using a rotational evaporator device such as a Buchi Rotavapor, spray drying, agitated thin film drying (“ATFD”), and the like.

Distillation can be conducted under vacuum, at elevated temperatures such as about 20° C. to about 70° C. Any temperature and vacuum conditions can be used as long as there is no increase in the impurity levels of the product.

Step iii) involves removing the protecting group from protected simvastatin amide of Formula V by reacting with an acid in presence of suitable organic solvent to afford simvastatin amide of Formula VI.

Suitable acids that can be used for the removal of the protecting group include but are not limited to organic and inorganic acids: organic acids include formic acid, acetic acid, p-toluene sulfonic acid and like; inorganic acid include sulphuric acid, hydrochloric acid and the like; and mixtures thereof or their combination in various propositions with water or with alcohol.

Suitable solvents that can be used for the removal of the protecting group include but are not limited to: C₁-C₄ straight chain alcohols such as methanol, ethanol, isopropyl alcohol, n-butanol, and the like; ketones such as acetone, ethyl methyl ketone, methyl isobutyl ketone, and the like; esters such as ethyl acetate, n-propyl acetate, n-butyl acetate, t-butyl acetate, and the like; nitriles such as acetonitrile, priopionitrile, and the like; ethers such as diethyl ether, dimethyl ether, tetrahydrofuran and the like; and mixtures thereof.

Suitable temperatures for the removal of the protecting group can range from −10 to 70° C., or from about 0 to 40° C., or the reflux temperature of the solvent used, either at atmospheric pressure or with another pressure. The duration of maintenance of the reaction mixture at the reaction temperatures for reaction completion vary considerably depending on the reactants and conditions chosen, for example about 1 to 48 hours, or about 3 to 36 hours, or about 4 to 12 hours, or longer.

The simvastatin amide of Formula VI prepared according to the present invention can be progressed into the next stage either after isolating or without isolating the compound.

In an embodiment, the simvastatin amide compound of Formula VI is progressed into the next stage without isolation.

Step iv) involves hydrolysis of simvastatin amide of Formula VI to form simvastatin acid of Formula VII, which is further converted into a simvastatin ammonium salt of Formula VIII in an intermediate step.

The hydrolysis of simvastatin amide to form simvastatin acid of Formula VII can be carried out by treating simvastatin amide of Formula VI with a base.

Suitable bases include but are not limited to: alkali metal hydroxides such as sodium hydroxide, potassium hydroxide and the like; and metal carbonates such as sodium carbonate, potassium carbonate and the like.

Simvastatin acid that is formed in the reaction is converted into simvastatin ammonium salt of Formula VIII by treating simvastatin acid with ammonium hydroxide in the presence of methanol as a solvent. The product can then be filtered and dried to afford the simvastatin ammonium salt compound of Formula VIII.

Step v) involves forming a lactone ring in the simvastatin ammonium salt compound of Formula VIII to afford simvastatin of Formula I.

Lactone formation is an equilibrium reaction. Several procedures have been developed in order to shift the equilibrium to lactone formation. According to the process of the present invention, this step can be carried out by heating a solution containing simvastatin ammonium salt in the presence of a catalytic amount of p-toluene sulphonic acid.

The solution of simvastatin ammonium salt can be prepared by dissolving ammonium salt of simvastatin in a suitable solvent.

Suitable solvents that can be used for the preparation of the solution include but are not limited to aromatic hydrocarbons such as benzene, toluene, xylene and the like; nitrile solvents such as acetonitrile, propionitrile and the like; and mixtures thereof.

Water that is formed as a by-product during the reaction can be removed continuously from the reaction mass by azeotropic reflux or by any other conventional method.

After the complete removal of the water, methanol is added to the obtained reaction product and optionally treated with activated charcoal to afford a clear solution.

The obtained simvastatin according to the present invention is than isolated after adding water to the solution as an antisolvent.

After the addition of antisolvent the compound of Formula I is isolated using techniques known in the art including decantation, vacuum filtration, gravity filtration, centrifugation and the like.

The obtained solid material is further dried using any technique such as fluid bed drying (FBD), spin flash drying, aerial drying, oven drying or other techniques known in the art at temperatures of about 40° C. to 75° C. with or without application of vacuum and/or under inert conditions. In an embodiment drying is carried out under vacuum at about 60° C.

Simvastatin obtained by the aforementioned process can be further purified by recrystallizing from a mixture of n-hexane and ethyl acetate.

Suitable temperatures for providing a solution may range from about 10 to 60° C., or about 20 to 50° C., or room temperature.

The simvastatin is subjected to cooling to a lower temperature of about 5° C. to 40° C., or from about 10° C. to 35° C., to precipitate the product, which can be isolated using techniques known in the art. Such technique includes but are not limited to decantation, filtration, centrifugation, distillation of the solvent and the like.

The obtained simvastatin can optionally be dried using any technique such as fluid bed drying (FBD), spin flash drying, aerial drying, oven drying or other techniques known in the art at temperatures of about 40° C. to 75° C. with or without application of vacuum and/or under inert conditions. In an embodiment drying is carried out under vacuum at about 60° C.

Simvastatin thus obtained can be further purified by adding water to a solution of simvastatin in methanol and recovering the solid product.

Finally, the obtained simvastatin can optionally be converted into a premix of simvastatin by treating simvastatin with butylated hydroxyanisole in the presence of methanol as solvent. This can conveniently be done by adding butylated hydroxyanisole to the water that is used for precipitating simvastatin from a methanol solution, or by adding butylated hydroxyanisole to a methanol solution of simvastatin and then adding the water. An intimate mixture of simvastatin and butylated hydroxyanisole is recovered in solid form.

In all the steps, the solutions of any intermediate may be purified by treatment with activated charcoal, silica gel, kieselguhr or other suitable materials; another suitable method of purification is crystallization from a proper solvent. Nevertheless, these purifications typically will be applied only for characterization purposes. Due to high conversion and low amounts of side products, all the reaction steps of the present invention proceed in practical production substantially without the need of purification.

The compounds of the present invention can exist in unsolvated as well as solvated forms, including hydrated forms. In general, the solvated forms are equivalent to the unsolvated forms for purpose of the invention.

Simvastatin prepared according to the process of the present invention contains: less than or equal to about 0.4% by weight of impurity A (simvastatin hydroxy acid); less than or equal to about 0.4% by weight of impurity B (simvastatin acetate ester); less than or equal to about 0.4% by weight of impurity C (anhydro simvastatin); less than or equal to about 0.4% by weight of impurity D (dimer of simvastatin); less than or equal to about 1% by weight of impurity E & F (lovastatin & epilovastatin); less than or equal to about 0.4% by weight of impurity G (methylene derivative); and less than or equal to about 1% of total impurities; as measured by high performance liquid chromatography (“HPLC”).

Simvastatin obtained by the process of the invention is analyzed using the HPLC method described in European Pharmacopoeia 5^{th} Edition, Vol 2, January 2005, at pages 2413 and 2415.

Simvastatin obtained by the process of the present invention contains less than about 5000 ppm, or less than about 3000 ppm, or less than about 1000 ppm, of total residual organic solvents and less than about 200 ppm, or less than about 100 ppm, or less than about 60 ppm, of individual residual organic solvents as determined by gas chromatography (“GC”).

Simvastatin obtained by the process of the present invention has particle sizes D₉₀ less than about 15 μm.

The D₁₀, D₅₀, and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value. Methods for determining D₁₀, D₅₀ and D₉₀ include laser diffraction, such as using equipment sold by Malvern Instruments Ltd. of Malvern, Worcestershire, United Kingdom.

Simvastatin obtained by the process of the present invention has a particle size distribution of D₁₀ less than or equal to about 1 μm, D₅₀ less than or equal to about 5 μm, and D₉₀ less than or equal to about 12 μm. There is no specific lower limit for any of the D values.

In another aspect the present invention provides the hydroxy-protected lovastatin compound of Formula III; 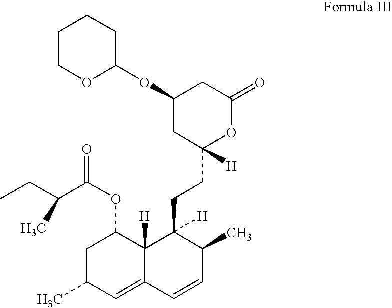
lovastatin amide of Formula IV; 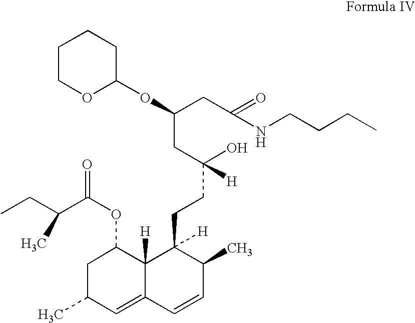
protected simvastatin amide of Formula V; and processes for their preparation.

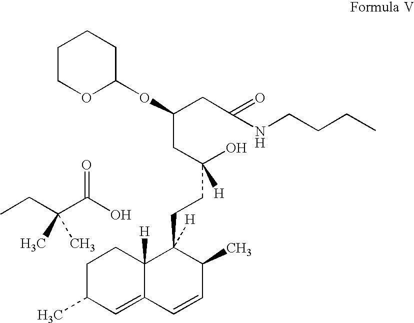

This process involves hydroxy group-protected derivatives of lovastatin as reaction intermediates, said derivatives having good stability, being easily synthesizable and allows preparation of the desired compounds in good yield and having good purity, wherein the use of expensive and unstable reactants for protection of the hydroxy group is substantially obviated.

The process of the present invention is eco-friendly, industrially well suited, commercially viable, reproducible and cost effective.

Certain specific aspects and embodiments of the invention will be explained in more detail with reference to the following examples, which are provided by way of illustration only and should not be construed as limiting the scope of the invention in any manner.

### EXAMPLE 1

### Preparation of Hydroxy-Protected Lovastatin Amide (Formula IV)

20 ml of tetrahydrofuran (THF) and 25 ml of 3,4-dihydropyran were placed into a round bottom flask and subjected to stirring for a period of 5 minutes. 5 ml of THF and 0.03 ml of sulphuric acid were added to the above solution over a period of about 10 minutes. 50 g of lovastatin was added to the obtained reaction solution and heated to a temperature of about 38° C. for a period of 25 minutes. 17 ml of n-butyl amine was added to the obtained reaction mass and it was heated to a temperature of 49° C. for a period of 2 hours. The reaction mixture was subjected to distillation at 45° C. by applying vacuum to afford 74 g of the title compound.

### EXAMPLE 2

### Preparation of Protected Simvastatin Amide (Formula V)

375 ml of THF was added to a round bottom flask and stirred, and 65 ml of pyrrolidine was added. The solution was cooled to −24° C. and at this temperature 419 ml of n-butyl lithium was slowly added over a period of about 70 minutes. The reaction mass was stirred for a period of about 10 minutes at −24° C. and further cooled to −44° C. 50 g of protected lovastatin amide from Example 1 dissolved in 280 ml of tetrahydrofuran was added to the above reaction mass at −44° C. and subjected to stirring for a period of 60 minutes. 39.8 ml of methyl iodide was added to the above reaction mass at −44° C. with stirring for a period of about 2.5 hours. The obtained reaction mass was then quenched by adding 500 ml of water at −10° C. and stirred for a period of about 35 minutes. The layers were separated and the organic layer was treated with 600 ml of 1 N aqueous HCl. The aqueous layer was saturated with 100 g of ammonium chloride and then was extracted with 200 ml of n-hexane. The organic layers were combined and subjected to vacuum distillation at 49° C. to afford 57 g of the title compound.

### EXAMPLE 3

### Preparation of Simvastatin Acid Ammonium Salt (Formula VIII)

50 g of protected simvastatin amide obtained from Example 2 and 100 ml of methanol were taken into a round bottom flask and stirred for a period of about 5 minutes. The solution was subjected to cooling to a temperature of about 2° C. at which temperature 10 ml of hydrochloric acid was added. The reaction was maintained at 10° C. to afford the compound simvastatin amide of Formula VI.

17 ml of sodium hydroxide solution was added to the above reaction mass and subjected to reflux in a water bath at 70° C. The reaction mass was cooled to 30° C. and 100 ml of water was added. The reaction mixture was stirred at 20° C. for a period of 10 minutes and the pH adjusted to 4.8 using 10% aqueous HCl. The reaction mass was stirred and the organic layer containing simvastatin acid of Formula VII was separated.

11.5 ml of ammonium hydroxide and 11.5 ml of methanol were added to the organic layer and subjected to stirring for a period of 30 minutes. The mass was cooled to 3° C. for 30 minutes, filtered and washed with 200 ml of precooled ethyl acetate. The wet material was subjected to suction drying for 30 minutes and finally dried at 45° C. to afford 21 g of the title compound.

### EXAMPLE 4

### Preparation of Simvastatin

172.5 liters of acetonitrile, 25 kg of simvastatin ammonium salt, 0.5 kg of p-toluenesulphonic acid and 402 liters of toluene were added into a reactor and subjected to heating at a temperature of 80° C. over a period of 29 hours. Water that was formed during the reaction was separated by azeotropic reflux. The reaction mass was cooled to a temperature of 42.5° C. and the reaction mass was filtered. The solvent was completely distilled from the filtrate at 60° C. under vacuum of about 650 mm Hg and than cooled to 45° C. 300 liters of methanol was added to the residue and stirred for a period of about 20 minutes. 2.5 kg of activated carbon was added to the obtained mass and subjected to stirring for a period of 30 minutes. The mass was filtered, washed with 50 liters of methanol and heated to 37.8° C. 350 liters of water was added to the filtrate and cooled to 12.5° C. over a period of 2 hours. The obtained solid mass was centrifuged followed by washing with 110 liters of precooled mixture of 55 liters of methanol and 55 liters of water. The wet material was subjected to spin drying for a period of 2 hours and subjected to drying at 54° C. Finally the obtained compound was cooled to 32° C. to afford 14.5 kg (yield: 63%) of the title compound.

Purification: 29 liters of ethyl acetate and 14.5 kg of simvastatin obtained above and 116 liters of n-hexane were taken into a reactor and heated to reflux at a temperature of 50° C. The reaction mass was than cooled to 32° C. over a period of 2 hours. The reaction mass was filtered and washed with 14.5 liters of n-hexane and dried for 1 hour. Thus the obtained wet material was dried at a temperature of about 55° C. and cooled to 34° C. to afford 11.5 kg (yield: 79.31%) of the title compound.

Loss on drying at 60° C.: 0.35% w/w.

Related substances by HPLC:

| | Content |
|---|---|
| Impurity | (weight %) |
| Hydroxy acid | 0.03 |
| Lovastatin | 0.55 |
| Methylene derivative | 0.1 |
| Acetate ester | 0.05 |
| Anhydro simvastatin | 0.05 |
| Dimer of simvastatin | 0.12 |
| Unknown impurity | 0.09 |
| Total Impurities | 1.46 |

### EXAMPLE 5

### Preparation of BHA Premix of Simvastatin

138 liters of methanol and 11.5 kg of simvastatin were taken into a reactor and subjected to stirring for complete dissolution. 1.15 kg of activated carbon was added to the solution and subjected to stirring for 30 minutes. The suspension was filtered through a 0.2 micron filter to make it particle free, washed with 23 liters of methanol and subjected to heating to 38° C. 161 liters of water was added over a period of 50 minutes and cooled to a temperature of 12.5° C. over a period of 2 hours. The obtained mass was centrifuged and washed with 25.3 liters of precooled water. The wet material was subjected to spin drying for 2 hours. 25.3 liters of methanol, 66 liters of water and 0.017 kg of butylatedhydroxyanisole were added into a reactor and stirred about 10 minutes. To the obtained solution the above spin-dried material was added and maintained for 30 minutes at 31° C. The obtained solid material was subjected to centrifugation and washed with 69 liters of water. The wet material was centrifuged for 2 hours and dried at 44° C. Finally the dried material was cooled to 30° C. to afford 10.3 kg (yield: 89.5%) of the title compound.

Loss on drying at 60° C.: 0.21% w/w

Related substances by HPLC:

| | Content |
|---|---|
| Impurity | (weight %) |
| Hydroxy acid | 0.010 |
| Lovastatin | 0.55 |
| Methylene derivative | 0.1 |
| Acetate ester | 0.01 |
| Anhydro simvastatin | 0.05 |
| Dimer of simvastatin | 0.1 |
| Unknown impurity | 0.01 |
| Total impurities | 0.3 |

Residual solvents by GC:

| | Content |
|---|---|
| Solvent | (ppm) |
| Hexane | 56 |
| Tetrahydrofuran | 2 |
| Ethyl acetate | 3 |
| Methanol, toluene and | Not Detected |
| acetonitrile | |

1. A process for preparing simvastatin, comprising reacting a compound having a formula: 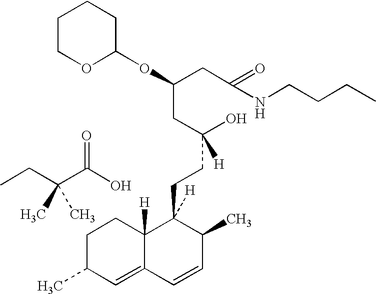
with an acid to form a compound having a formula:

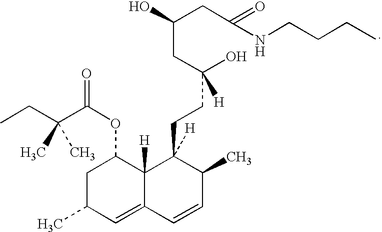

2. A process for preparing simvastatin, comprising methylating a compound having a formula: 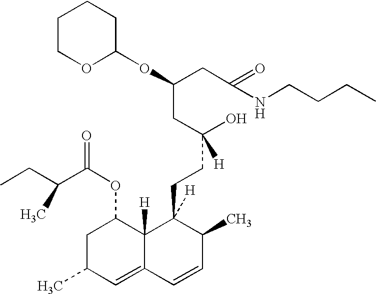
to form a compound having a formula:

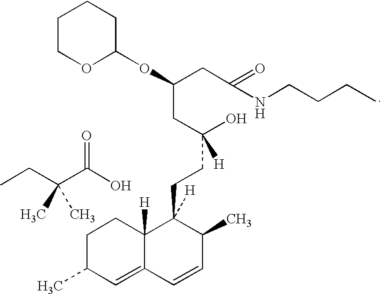

3. A process for preparing simvastatin, comprising reacting lovastatin with 3,4-dihydro-2H-pyran to form an intermediate having a formula: 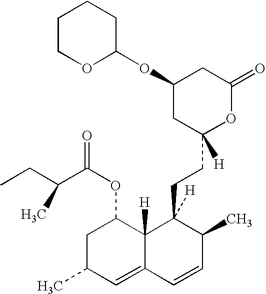
and, without isolating an intermediate, reacting with an amine to form a compound having a formula:

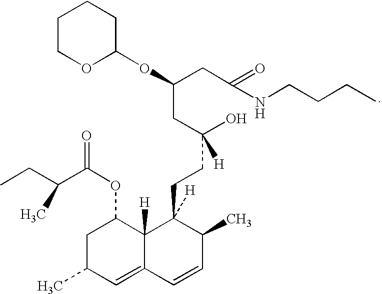

4. A process for preparing simvastatin, comprising reacting lovastatin with 3,4-dihydro-2H-pyran to form an intermediate having a formula: 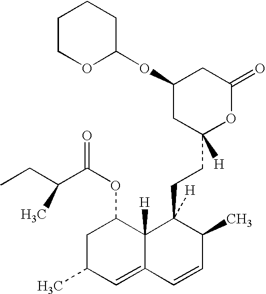
and, without isolating an intermediate, reacting with an amine to form a compound having a formula: 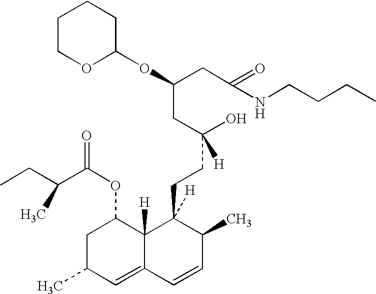
then methylating to form a compound having a formula: 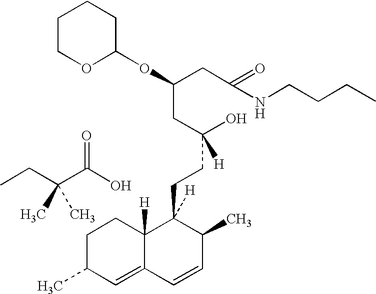
and reacting with an acid to form a compound having a formula:

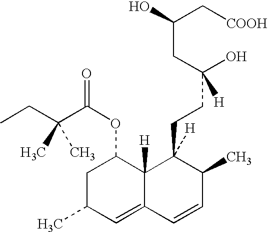

5. The process of claim 4, wherein a compound having a formula: 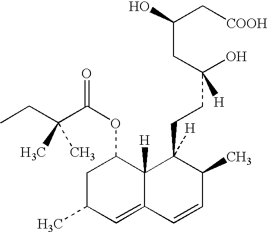
is not isolated prior to further reaction.

6. The process of claim 4, further comprising hydrolyzing a compound having a formula: 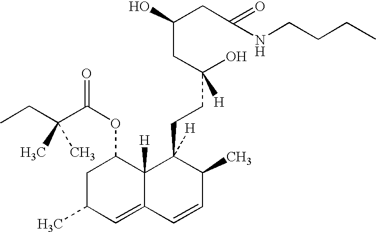
to form an acid, reacting with ammonia to form a salt, and then forming a lactone ring.

7. The process of claim 4, further comprising hydrolyzing a compound having a formula: 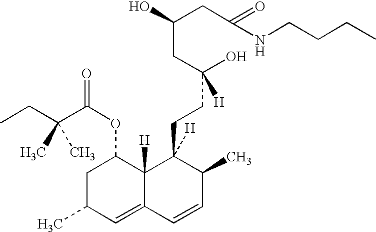
to form an acid having a formula: 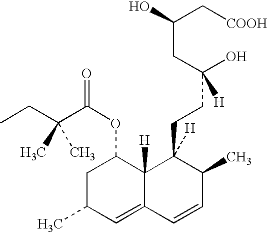
and, without isolating an acid, reacting with ammonia to form a salt, and then forming a lactone ring.

8. A compound having a formula:

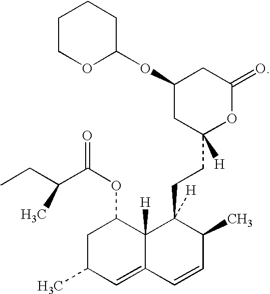

9. A compound having a formula:

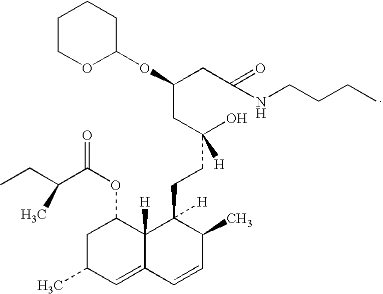

10. A compound having a formula:

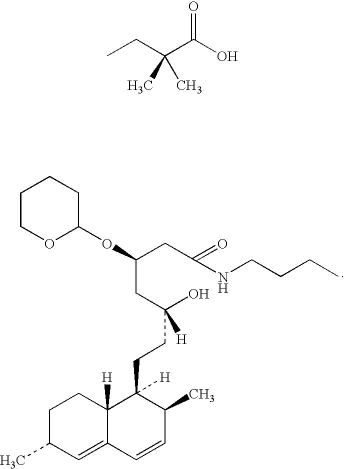

11. A solid premix comprising simvastatin and butylated hydroxyanisole.

12. A process for preparing a solid premix, comprising combining a solution of simvastatin with butylated hydroxyanisole and water.

13. The process of claim 12, wherein a solution comprises methanol as a solvent.

14. The process of claim 12, wherein a premix is formed by adding a mixture comprising butylated hydroxyanisole and water to a solution of simvastatin.

15. The process of claim 14, wherein a solution comprises methanol as a solvent.

16. The process of claim 12, wherein a premix is formed by adding water to a solution comprising simvastatin and butylated hydroxyanisole.

17. The process of claim 16, wherein a solution comprises methanol as a solvent.
